(19) United States
(12) Patent Application Publication
(10) Pub. No.: US 2017/0369597 A1
(43) Pub. Date: Dec. 28, 2017

(54) PHYTOGLYCOGEN NANOPARTICLES AND METHODS OF MANUFACTURE THEREOF USING CORN

(71) Applicant: MIREXUS BIOTECHNOLOGIES INC., Guelph (CA)

(72) Inventors: Anton KORENEVSKI, Guelph (CA); Erzsebet PAPP-SZABO, Guelph (CA); John Robert DUTCHER, Guelph (CA); Oleg STUKALOV, Kitchener (CA); Rokshana NAZNIN, Guelph (CA); Khalid MOOMAND, Brantford (CA)

(21) Appl. No.: 15/644,333

(22) Filed: Jul. 7, 2017

Related U.S. Application Data
(63) Continuation-in-part of application No. 14/787,207, filed on Oct. 26, 2015, now Pat. No. 9,737,608.
(60) Provisional application No. 61/816,686, filed on Apr. 26, 2013.

(51) Int. Cl. C08B 37/00; B82Y 5/00; G01N 21/47

(52) U.S. Cl. CPC C08B 37/009; B82Y 5/00; G01N 21/47

(57) ABSTRACT

An industrially scalable process for producing substantially monodisperse compositions of phytoglycogen nanoparticles from phytoglycogen-containing plant materials is provided that avoids the use of chemical, enzymatic or thermo treatments that degrade the phytoglycogen material. Also provided are phytoglycogen nanoparticle compositions produced by these processes.

### CROSS-REFERENCE TO RELATED APPLICATIONS

This application is a continuation in part of U.S. patent application Ser. No. 14/787,207, which claims priority from U.S. patent application 61/816,686 filed Apr. 26, 2013 and the contents of these applications are incorporated herewith in their entirety.

### TECHNICAL FIELD

This invention relates to phytoglycogen nanoparticles and methods of producing phytoglycogen nanoparticles using sweet corn.

### BACKGROUND OF THE ART

Phytoglycogen and glycogen are polysaccharides of glucose composed of α-1,4-glucan chains, highly branched via α-1,6-glucosidic linkages, which function as energy storage mediums in plant and animal cells. Glycogen is present in animal tissue in the form of dense particles with diameters of 20-200 nm. Glycogen is also found to accumulate in microorganisms, e.g., bacteria and yeasts. Phytoglycogen is a polysaccharide that is similar to glycogen, both in terms of its structure and physical properties and originates in plants.

Glycogen and phytoglycogen are considered “highly polydisperse” or heterogeneous materials. Glycogen typically has a molecular weight between 10⁶ and 10⁸ Daltons with a corresponding large polydispersity for known preparations. Transmission electron microscopy (TEM) observations of animal and plant tissues and extracted glycogen/phytoglycogen preparations have revealed the particulate nature of these polysaccharides. Commonly reported glycogen or phytoglycogen particle diameters are in the range of 20-300 nm and have either continuous or multimodal size distribution. Small, 20-30 nm, particles are termed β-particles and large, 100-300 nm—α-particles. The α-particles are considered to be composed of β-particles as a result of aggregation or clustering [1].

Various methods have been developed to isolate glycogen and phytoglycogen from living organisms, most often for the purpose of quantifying the amount of total glycogen accumulated in biological samples, and, infrequently, for the purpose of using the glycogen as a product in applications.

The most frequently used method is extraction from animal tissues, particularly from marine animals, especially mollusks, because of their ability to accumulate glycogen. For example, U.S. Pat. No. 5,734,045 discloses a process for the preparation of protein-free glycogen from mussels by using hot alkali extraction, following neutralization and treatment of the resulting solution with cationic resins. Glycogen can also be produced via fermentation of yeasts as described, for example, in patent application WO/1997/021828. U.S. Pat. No. 7,670,812 describes a process for the biosynthetic production of glycogen-like polysaccharides by exposing a mixture of enzymes to low molecular weight dextrins. Sweet corn and sweet rice can be used as a source of glycogen; see, for example, patent application EP0860448B1, which describes a process of isolating glycogen from the kernels of sweet rice.

The main steps of glycogen/phytoglycogen isolation typically include: biomass disintegration via pulverization/grinding/milling etc.; glycogen extraction into water phase; separation of insoluble solid particles via filtration and/or centrifugation; elimination of finely dispersed or solubilized lipids, proteins and low molecular weight contaminates; and concentration and drying.

To increase the yield of glycogen in the second extraction step, extraction is often performed at elevated temperatures and/or using alkaline or acidic solutions. Such procedures include initial treatment of ground biological material with hot concentrated (20-40%) solution of alkali [2, 3], cold acids [4] or boiling water [5].

The procedures used in the conventional methods of glycogen isolation/purification result in considerable hydrolysis of the glycogen structure, with significant increases of lower molecular weight products and chemical alteration of the molecule.

Various milder extraction procedures have been developed, such as cold water extraction [6], and resulting products were claimed to be close representation of natural state of the glycogen. However, known glycogen preparations produced by cold water extraction method are highly polydisperse [7,1].

Various methods are known for performing the step of purifying crude glycogen extract from finely dispersed proteins, lipids, nucleic acids, and other polysaccharides. Protein and nucleic acids can be removed via selective precipitation with deoxycholate (DOC) trichloroacetic acid (TCA), polyvalent cations, and/or enzymatic (protease, nuclease) treatment. Also methods of removing proteins by salting them out (e.g., with ammonium sulfate), or by ion-exchange have been used. Another common method of protein removal is thermal coagulation, normally at 65-100° C., following by centrifugation or filtration. Autoclaving (121° C. at 1 atm) has also been used to coagulate proteins in phytoglycogen extract [8]. Furthermore, proteins and lipids can be removed with phenol-water extraction.

International patent application publication no. WO 2013/019977 (Yao) teaches a method for obtaining extracts that include phytoglycogen that includes ultrafiltration, but also subjecting the aqueous extract to enzymatic treatment that degrades both phytoglycogen as well as other polysaccharides. Yao provides a method to reduce viscosity of phytoglycogen material by subjecting it to beta-amylolysis, i.e., enzymatic hydrolysis using beta-amylase. The “purified phytoglycogen” materials yielded by the methods of Yao include not only phytoglycogen, but derivatives of phytoglycogen, including beta-dextrins and the digestion products of other polysaccharides. The method of Yao further involves heating the extract to 100° C. (see Yao Example 1).

U.S. Pat. No. 5,895,686 discloses a method for extracting phytoglycogen from rice by water or a water-containing solvent (at room temperature) and the removal of proteins by thermal denaturation and TCA precipitation. The product has a multimodal molecular weight distribution, with correspondingly high polydispersity, and large water solution viscosities. These properties can be attributed to the presence of substantial amounts of amylopectin and amylose in glycogen preparations from plant material, but also to glycogen degradation during the glycogen extraction process.

U.S. Pat. Nos. 5,597,913 and 5,734,045 describe procedures that result in glycogen that is substantially free of nitrogenous compounds and reducing sugars as an indication of its purity from proteins and nucleic acids. These patents teach the use of boiling of the selected tissues in solutions of high pH.

United States patent application publication no. United States 20100272639 A1, assigned to the owner of the present invention, provides a process for the isolation of glycogen from bacterial and shell fish biomass. Bacteria is taught as preferred since the process can be conducted to yield a biomass that does not have other large molecular weight polysaccharides such as amylopectin and amylose and is free of pathogenic bacteria, parasites, viruses and prions associated with shell-fish or animal tissue. The processes disclosed generally include the steps of cell disintegration by French pressing, or by chemical treatment; separation of insoluble cell components by centrifugation; elimination of proteins and nucleic acids from cell lyzate by enzymatic treatment followed by dialysis which produces an extract containing crude polysaccharides and lypopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and freeze drying to produce a powder of glycogen. Glycogen isolated from bacterial biomass was characterized by MWt 5.3-12.7×10⁶ Da, had particle size 35-40 nm in diameter and were monodisperse (PDI=Mₙ/M_{w}=1.007-1.03).

There remains a need for processes of producing phytoglycogen products that are scalable while yielding high quality product.

### BRIEF SUMMARY

In one embodiment, there is provided a process for producing monodisperse phytoglycogen nanoparticles that includes: a. subjecting disintegrated phytoglycogen-containing plant material to a water treatment at a temperature equal to or less than about 70° C.; b. adjusting the pH of the product of step (a.) to between about 3.5 and about 7; c. subjecting the product of step (b.) to a solid-liquid separation to obtain an aqueous extract; and d. subjecting the aqueous extract from step (c.) to ultrafiltration to remove impurities having a molecular weight of less than about 300 kDa to obtain an aqueous composition comprising monodisperse phytoglycogen nanoparticles. The disintegrated phytoglycogen-containing plant is prepared using a disintegration process selected to minimize or avoid emulsification of lipids and proteins found in the plant material. The process steps are also implemented so as to minimize emulsification of lipids and proteins.

In one embodiment, the phytoglycogen-containing plant material is corn kernels, which may be frozen. In one embodiment, the phytoglycogen-containing plant material is standard type (su) or surgary extender (se) type sweet corn. In one embodiment, the phytoglycogen-containing plant material is milk stage or dent stage kernel of standard type (su) or surgary extender (se) type sweet corn.

In one embodiment, the aqueous composition of monodisperse phytoglycogen nanoparticles of step (d.) is subjected to enzymatic treatment using amylosucrose, glycosyltransferase, branching enzymes or any combination thereof.

In one embodiment, the ultrafiltration of step (d.) removes impurities having a molecular weight less than about 500 kDa. In one embodiment, the process further includes (d1) passing the aqueous extract of step (c.) through microfiltration material having a maximum average pore size between about 10 μm and about 40 μm. An adsorptive filtration aid may be added prior to step (d.) or step (d1.), which may be a diatomaceous earth.

In one embodiment, the solid-liquid separation involves gently agitating the product of step (b.) for a period of 30 to 60 minutes. In this context, gently agitating refers to agitating so as to avoid or minimize emulsification of lipids and proteins.

In one embodiment, the pH is adjusted using any suitable alkali, in one embodiment NaOH. In one embodiment, the pH is adjusted using any suitable acid or combination of acids, which may, for example, be one or more of phosphoric acid, acetic acid, citric acid, sulfuric acid and hydrogen chloride.

In one embodiment, the process further includes (e.) drying the aqueous composition comprising monodisperse phytoglycogen nanoparticles to yield a dried composition of substantially monodisperse phytoglycogen nanoparticles.

Also provided is a composition produced according to the processes described above of phytoglycogen nanoparticles obtained from a phytoglycogen-containing plant material, the phytoglycogen nanoparticles having a polydispersity index of less than 0.35 as measured by dynamic light scattering (DLS).

In some embodiments, the phytoglycogen nanoparticles have an average particle diameter of between about 30 nm and about 150 nm, between about 50 nm and 120 nm or between about 60 nm and 80 nm as measured by DLS.

In one embodiment, the phytoglycogen nanoparticles have a total protein content of 2-3% (w/w) as measured by the Bradford assay.

In one embodiment, the phytoglycogen nanoparticles have a total protein content of less than 0.1% (w/w) as measured by the Bradford assay.

In one embodiment, the phytoglycogen nanoparticles have a reducing sugar content of less than 0.15% as measured by the potassium ferricyanide calorimetric assay.

The composition may be a powder or an aqueous dispersion of the phytoglycogen nanoparticles.

### BRIEF DESCRIPTION OF THE DRAWINGS

FIG. 1 is a schematic drawing of a phytoglycogen nanoparticle.

FIG. 2 shows particle size distribution of the phytoglycogen isolated according to Example 1 using DLS.

FIG. 3 shows particle size distribution of the phytoglycogen isolated according to Example 2 using DLS.

FIG. 4 shows viscosity versus concentration (w/w %) for a dispersion of monodisperse phytoglycogen nanoparticles in water according to an embodiment of the present invention.

FIG. 5 shows the extent of pulverisation of the corn kernel and its impact on the quality of extraction in Step (c) as described in Example 5.

FIG. 6 shows the protein content of isolated phytoglycogen according to Example 7 as measured by the Bradford assay in Example 11.

FIG. 7 shows the visual differences between extracting phytoglycogen without pH adjustment and pH adjustment during incubation and as a final powdered product according to Example 8.

FIG. 8a shows particle size distribution using DLS of isolated phytoglycogen according to Example 10 where the pH was unadjusted according to Example 8.

FIG. 8b shows particle size distribution using DLS of isolated phytoglycogen according to Example 10 where the pH was mildly acidified according to Example 8.

FIG. 9 shows the protein content of isolated phytoglycogen according to a series of pH adjustments as measured by the Bradford assay in Example 11.

FIG. 10 shows the lipid content of isolated phytoglycogen according to Example 8 as measured according to Example 12.

FIG. 11 shows the reducing sugar content of isolated phytoglycogen according to Example 8 as measured according to Example 13.

FIG. 12 shows the size exclusion properties of phytoglycogen nanoparticles prepared as in Example 8, where Fig (a) represents unadjusted pH and Fig (b) acidified pH.

### DETAILED DESCRIPTION

“Phytoglycogen” as used herein refers to a nanoparticle of α-D glucose chains obtained from plant material, having an average chain length of 11-12, with 1→4 linkage and branching point occurring at 1→6 and with a branching degree of about 6% to about 13%.

In one embodiment, there is provided a composition of monodisperse nanoparticles of a high molecular weight glucose homopolymer. In one embodiment, there is provided a composition of monodisperse phytoglycogen nanoparticles.

The polydispersity index (PDI), determined by dynamic light scattering (DLS) technique, is defined as the square of the ratio of standard deviation to mean diameter: PDI=(σ/d)². Also, PDI can be expressed through the distribution of the molecular weight of polymer, and defined as the ratio of M_{W} to Mₙ, where M_{w} is the weight-average molar mass and Mₙ is the number-average molar mass (hereafter this PDI measurement is referred to as PDI*). In the first case, monodisperse material has PDI close to zero (0.0), and in the second—1.0. In one embodiment, there is provided a composition of phytoglycogen nanoparticles having a PDI of less than about 0.35, less than about 0.3, less than about 0.2, less than about 0.17, less than about 0.15, less than about 0.10, less than 0.07 or less than 0.05 as measured by DLS. In one embodiment, there is provided a composition of phytoglycogen nanoparticles having a PDI* of less than about 1.35, less than about 1.3, less than about 1.2, less than about 1.17, less than about 1.15, less than about 1.10, or less than 1.05 as measured by SEC MALS.

Monodispersity is advantageous for a number of reasons, including that surface modification and derivatization occurs much more predictably if the nanoparticles of a composition are monodisperse. Size also affects the distribution and accumulation of the nanoparticles in biological tissues, as well as pharmacokinetics. Furthermore, narrow size distribution is critical for such applications as diagnostic probes, catalytic agents, nanoscale thin films, and controlled rheology.

Nanoparticle size, including distributions (dispersity) and average values of the diameter, can be measured by methods known in the art. These primarily include DLS and microscopy techniques, e.g. TEM, and atomic force microscopy.

In one embodiment, there is provided a monodisperse composition of phytoglycogen nanoparticles having an average particle diameter of between about 30 and about 150 nm as measured by DLS, in one embodiment, between about 50 nm and about 120 nm, and in one embodiment, between about 60 nm and 80 nm. These nanoparticles are individual particles as opposed to clustered α-particles seen in prior compositions.

The phytoglycogen is suitably produced from corn.

Varieties used must be phytoglycogen-containing. Whether a variety contains phytoglycogen can be readily determined by those of skill in the art using known techniques. In addition, for many varieties, published literature identifies whether a variety contains phytoglycogen.

In one embodiment, the composition is obtained from sweet corn (Zea mays var. saccharate and Zea mays var. rugosa). In one embodiment, the sweet corn is of standard (su) type or sugary enhanced (se) type.

In one embodiment, the composition is obtained from dent stage or milk stage kernels of sweet corn.

In one embodiment, the monodisperse composition of phytoglycogen nanoparticles is substantially pure. In various embodiments, the composition based on dry weight is composed of at least about 80%, at least about 85%, at least about 90%, at least about 95%, or at least about 99% phytoglycogen nanoparticles having a diameter size of between about 30 nm and about 150 nm as measured by DLS. In one embodiment, the composition based on dry weight is composed of at least about 80%, at least about 85%, at least about 90%, at least about 95% or at least about 99% phytoglycogen nanoparticles having a diameter size of between about 50 nm and about 120 nm as measured by DLS. In another embodiment, the composition based on dry weight is composed of at least 80%, at least about 85%, at least about 90%, at least about 95%, or at least about 99% phytoglycogen nanoparticles having a diameter size between about 60 nm and about 80 nm as measured by DLS.

In one embodiment, the composition is substantially free of other polysaccharides. In one embodiment, the composition contains less than about 10% of other polysaccharides. In one embodiment, the composition contains less than about 5% other polysaccharides. In one embodiment, the composition contains less than about 1% of other polysaccharides.

Glycogen

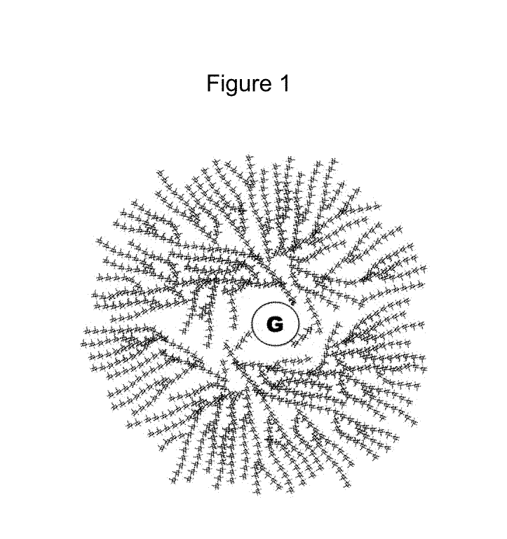

Glycogen and phytoglycogen consists of linear chains of glucose residues connected by α-1→4-glycosidic bonds, with branches that are attached through α-1→6-glycosidic bonds. Chemical analysis of mammalian glycogen from different sources suggests that its average chain length is ˜13 residues [9]. As shown in FIG. 1, an accepted model for glycogen structure has inner chains, which would normally contain two branch points, and outer chains, which are unbranched. The entire tree-shaped polymer is rooted in a single molecule of the protein glycogenin (G).

Density of the glycogen molecule increases exponentially with the number of tiers. It has been calculated that addition of a 13th tier to a glycogen molecule would add an impossible density of glucose residues, making 12 tiers a theoretical maximum [9]. An important feature is that the outermost tier of any molecule completely formed in this way would contain ˜45-50% of the total glucose residues of the molecule as unbranched A-chains, while the first eight inner tiers only contain ˜5% of the total glucose. Therefore a full-size glycogen molecule in this model would consist of 12 tiers, for a total of ˜53000 glucose residues, a molecular mass of ˜107 kDa and a diameter of ˜25 nm. Although predominantly composed of glucose residues, glycogen may contain other trace constituents, notably glucosamine and phosphate [1]. Mathematical modeling showed that the structural properties of the glycogen molecule depend on three parameters, namely, the branching degree (r), the number of tiers (t), and the number of glucose residues in each chain (gc) [9, 10, 11, 12, 13].

Despite the spherical shape of the glycogen molecule suggested by the mechanism of growth, on growing the molecule beyond a certain limit, it loses structural homogeneity, as the branching degree and the chain length degenerate in the last tiers.

Phytoglycogen

Although glycogen and phytoglycogen have very similar structure there is a principal difference in the functional purpose of these polysaccharides. Glycogen in animals and bacteria is meant to serve as a short-term “fuel” storage optimized for the fast turnover.

In plants the main energy source is starch, which is stored in leaves, stems, seeds, roots, etc. In contrast to glycogen, starch is a long-term energy strategy that allows the plant to survive during adverse climate situations. Starch contains two types of polyglucans: amylopectin (which is highly branched) and amylose (which is almost linear with few branches. There are large variations in the contents of the two components in starches from different sources, but amylopectin is commonly considered the major component in storage starch and accounts for about 65-85% by weight.

Phytoglycogen has a high molecular density in aqueous dispersions as a result of its highly branched structure. The dispersed molecular density of phytoglycogen from maize is over 1000 g/mol·nm³ compared to approximately 40 g/mol/nm³ for amylopectin [14], making phytoglycogen structurally robust and ideal for functional grafting at its surface. In contrast to amylopectin molecules, phytoglycogen molecules do not have long chains connecting individual clusters [15, 16]. The average chain length of phytoglycogen ranges from DP (degree of polymerization) 11-12 and branch density (i.e., the percentage of α-1,6 glucosidic linkages) of about 8-9%; a noticeable contrast to the average chain length of 17-18 and branch density of 6% for amylopectin [15, 17].

The high digestibility, low viscosity and surface functionality of phytoglycogen nanoparticles make them suitable as a delivery carrier of active ingredients. Despite the evident advantages of using phytoglycogen in food and nutraceuticals, cosmeceuticals and pharmaceutical applications, the cost prohibitive nature of producing a highly purified and industrially scalable phytoglycogen remains to date a significant challenge.

Amylopectin is synthesized by multiple isoforms of four classes of enzymes: soluble starch synthase (SS), starch branching enzyme (BE), ADPglucose pyrophosphorylase, and starch debranching enzyme (DBE). These are the same 4 classes of enzymes that are involved in glycogen synthesis.

This explains the similarity between amylopectin and glycogen structure: both are α-1,4-polyglucans with α-1,6-branching. However, the average chain length (g_{c}) in amylopectin is 20-25, about twice longer than in glycogen, while the degree of branching (r) is about 1.5-2 times lower.

Mutation in isoamylase (ISA) and, therefore, deficiency in debranching activity, results in partial substitution of amylopectin with phytoglycogen. Most common examples of such phytoglycogen accumulating plants are sugary 1 (su) mutants of corn, rice and other cereals.

Phytoglycogen structurally is similar to glycogen, having average chain length 11-12 and similar degree of branching and typically has a molecular weight between 10⁶-10⁸ Da. However, reported larger particle sizes than glycogen suggest lower degree of branching and/or lower structural homogeneity. Lower structural homogeneity of phytoglycogen is not unexpected, considering that glycogen is a highly optimized metabolic product, while phytoglycogen is a result of a derangement in amylopectin synthesis.

The present inventors have experimentally determined that the reported polydispersity of compositions of phytoglycogen is in fact partly due to destructive isolation methods, and observed polydispersity can further arise from the presence of finely dispersed contaminants such as proteins, lipids and other polysaccharides. Using methods described herein, the present inventors have produced monodisperse compositions of phytoglycogens.

Method of Producing Monodisperse Phytoglycogen

As discussed above, the main steps of glycogen/phytoglycogen isolation typically include: 1. Biomass disintegration via pulverization/grinding/milling etc.; 2. Glycogen extraction into water phase; 3. Separation of insoluble solid particles (solids) via filtration and/or centrifugation; 4. Elimination of finely dispersed or solubilized lipids, proteins and low molecular weight contaminates; and 5. Concentration and Drying. Some operations can be combined e.g., milling and extraction.

In Examples 1 and 2, the inventors provide methods of producing monodisperse phytoglycogen nanoparticles, which are characterized in Examples 3 and 4. These exemplified methods include (i) immersing a disintegrated phytoglycogen containing corn material in water at a temperature between about 0 and about 50° C.; (ii) subjecting the product of step; (i) to a solid-liquid separation to obtain an aqueous extract; (iii) passing the extract of step (ii) through a microfiltration material having a maximum pore size of between about 0.05 and 0.15 μm; and subjecting the filtrate of step (iii) to an ultrafiltration step.

While these processes enable the production of substantially monodisperse compositions of phytolglycogen nanoparticles the methods are most suitable for production at a laboratory scale. The present inventors have further developed processes for production of phytoglycogen nanoparticles from a sweet corn starting material that can readily be used industrially for production in large quantities having a high yield relative to the amount of treated raw material. These processes enable the extraction of the soluble phytoglycogen moiety void of corn's principal protein, zein, and lipid fractions present in the endosperm (and thus to obtain phytoglycogen nanoparticles in a non-hydrolyzed form) without the use of precipitating agents such as deoxycholate (DOC), tricholoracetic acid (TCA), polyvalent cations, and/or enzymatic (protease and nuclease) treatment thereby enabling the production of high quality monodisperse phytoglycogen nanoparticle compositions on an industrial scale.

Following a series of failed attempts to reduce residual contaminants of proteins, lipids and reducing sugars in the isolated phytoglycogen, this problem was surprisingly solved according to the industrially scalable methods described herein, which involve: taking a given quantity of sweet corn kernels and subjecting it to a gentle milling phase; extraction of the phytoglycogen nanoparticles into a temperature controlled and pH adjusted aqueous phase over a defined period of time; separation of insoluble solids via solid-liquid separation techniques such as centrifugation and filtration, and concentration and drying without the need for precipitation with one or more organic solvents.

In one embodiment, a method of producing monodisperse phytoglycogen nanoparticles is provided, which may be suitably practiced on an industrial scale. This method includes:

a. subjecting disintegrated phytoglycogen-containing plant material, in one embodiment gently milled corn kernels, to a water treatment at a temperature not exceeding 70° C.;

b. adjusting the pH of the product of step (a) to between 3.5 and 7;

c. subjecting the product of step (b.) to a solid-liquid separation to obtain an aqueous extract;

d. subjecting the aqueous extract from step (c.) to ultrafiltration to remove impurities having a molecular weight of less than about 500 kDa, to obtain a composition comprising substantially monodisperse phytoglycogen nanoparticles.

Suitably, in the water treatment of step a. the disintegrated phytoglycogen-containing plant material (for example corn kernels) are immersed in the temperature-controlled water.

While in one embodiment, in step (b.) the pH is adjusted to between about 3.5 and about 7, in further embodiments, the pH is adjusted to between about 4.5 and about 6, and between about 4.5 and about 5.5.

In one embodiment, the ultrafiltration of step d. is the sole filtration step in the method.

This method can be readily practiced on an industrial scale enabling the production of a highly purified phytoglycogen nanoparticle composition. This method is suitable for the production of large quantities of phytoglycogen, without the use of organic solvents. For example, the process may suitably be applied to a starting quantity of corn kernels of hundreds of kilograms or more (e.g. a tonne or more) and, with appropriate facilities, a daily yield of 100 tonnes of phytoglycogen nanoparticles is possible. In specific embodiments, phytoglycogen is extracted from sweet corn, particularly sweet corn kernels. In one embodiment frozen corn kernels.

The present inventors have surprisingly found that it is not necessary to thaw the corn kernels before practicing the processes as described herein as might be expected based on previous methods. In one embodiment, frozen kernels are gently disintegrated as described herein and the material is then allowed to thaw over the course of the process.

The aqueous dispersion can then be dried to yield a composition of substantially monodisperse phytoglycogen nanoparticles.

In one embodiment, the plant material is the kernel of sweet corn (Zea mays var. saccharate and Zea mays var. rugosa). In one embodiment, milk stage or dent stage maturity kernel of sweet corn is used.

The yield of phytoglycogen nanoparticles is in various embodiments, between about 5% and 50%, between about 10% and about 50%, between about 20% and 50%, between about 30% and about 50%, between about 40% and about 50%, between about 10% and about 40%, between about 20% and 40%, between about 30% and about 40% of the dry weight of the plant material. The exact yield of phytoglycogen will depend on the plant material used, including the variety and stage of maturity. In the case of corn, the inventors have obtained yields in the range of 35-40% of the kernel dry weight for milk stage kernel maturity and 20-30% for the dent stage maturity. These yields of monodisperse phytoglycogen were unexpected, given the high polydispersity of previously reported phytoglycogen.

Methods of preparing disintegrated plant material are known to those skilled in the art, e.g. grinding, milling or pulverizing of biomaterial. Regardless of the method used, it should be gentle. In particular, care should be taken to adjust the disintegration parameter to balance the need to rupture the hull (which enables release of phytoglycogen), while avoiding rupture of the germ (which contains corn oil). Further, disintegration parameters should be adjusted to minimize emulsification of proteins and lipids. These parameters can be readily determined empirically, as will be known by persons of skill in the art. The plant materials are suitably disintegrated to an average particle size of less than about 0.5 mm. In the method described, the disintegrated phytoglycogen-containing material is preferably prepared by gentle milling. In one embodiment, the water extraction of step (a.) is performed by agitating in a water bath the milled plant material for 30-60 mins. In one embodiment, the water extraction is performed at a temperature of between about 0° C. and about 70° C. In one embodiment, the water extraction is performed at a temperature of between about 0° C. and about 50° C., about 40° C., about 30° C. or about 20° C. The optimal period of agitation, temperature and agitation rate depend on the nature of the disintegrated biomass, and determining the same is within the purview of a person of skill in the art. While in one embodiment, the manner of adjusting the ph in step (b) is not particularly restricted. In one embodiment, the extract pH is adjusted with NaOH in process step (b). In another embodiment, the extract pH is adjusted with one or many of the following acids: phosphoric acid, acetic acid, citric acid, sulfuric acid, and hydrogen chloride in process step (b).

In one embodiment, a multistage filtration and ultrafiltration are performed, which eliminates most of the proteins, lipids and contaminating polysaccharides, including amylose and amylopectin, without any chemical, enzymatic or thermo treatment, thereby yielding a composition of monodisperse phytoglycogen nanoparticles.

When performed, microfiltration (which may be referred to as step (d1) may be performed in stages. However, the present inventors have found that the method may suitably be practiced by passing the aqueous extract through a single microfiltration material having a maximum pore size between about 10 μm and about 40 μm, in one embodiment, between about 15 μm and about 35 μm, in one embodiment, between about 20 μm and about 30 μm, and in one embodiment, about 25 μm. Accordingly, in one embodiment, the process involves a single microfiltration step and a single ultrafiltration step as described.

In one embodiment, an adsorptive filtration aid such as diatomaceous earth can be added to phytoglycogen extract. In one embodiment, the adsorptive filtration aid is used in an amount of about 2-10% wt/vol, in one embodiment, between about 3-5% wt/vol.

The aqueous extract from step (c.) (or the final filtrate from the microfiltration when this step is performed) is subject to ultrafiltration, which removes low molecular weight contaminants therefrom including salts, proteins and sugars e.g. dextrins, glucose, sucrose or maltose. Ultrafiltration is suitably performed by Cross Flow Filtration (CFF) with a molecular weight cut off (MWCO) of about 300 to about 500 kDa.

Various methods of microfiltration and ultrafiltration are known to those of skill in the art and any suitable method may be employed.

Optionally, following ultrafiltration the aqueous dispersion containing phytoglycogen can be subject to enzymatic treatment to reduce polydispersity. Suitably, it can be treated with amylosucrose, glycogen synthase, glycosyltransferase and branching enzymes or any combination thereof. However, enzymes that digest amylopectin and amylose (e.g. beta-amylase) should be avoided as they will yield a solution of polyglucans variously degraded, rather than a purified composition of phytoglycogen nanoparticles.

However, the present inventors have found that monodisperse and highly purified product may be obtained without the need for enzymatic treatment and thus in one embodiment the method is practiced without this optional step.

Phytoglycogen dispersions can be concentrated (up to 30%) by the process of CFF ultrafiltration. Alternatively, following CFF ultrafiltration, phytoglycogen can be precipitated with a suitable organic solvent such as acetone, methanol, propanol, etc., preferably ethanol, although in a preferred embodiment, the method is practiced without the use of organic solvents.

The method further includes drying the phytoglycogen extract, suitably by spray drying or freeze drying. Various standard concentrating and/or drying methods, such as use of a falling film evaporator, a rising film evaporator, spray drying, freeze drying, drum drying, or combinations thereof, etc., can be used to dehydrate the phytoglycogen dispersion and/or collect the solid form of phytoglycogen product.

In one embodiment, the phytoglycogen nanoparticles have a total protein content of 2-3% (w/w) as measured by a spectrophotometric method familiar to those practicing the art (in particular, through the use of Bradford assay). In one embodiment, the phytoglycogen nanoparticles have a total protein content less than 0.1% (w/w).

In one embodiment, the phytoglycogen nanoparticles produced by methods as described herein have a reducing sugar content of less than 0.15% as measured by the potassium ferricyanide colorimetric assay.

Chemical Functionalization of the Nanoparticles

Embodiments of the present invention include nanoparticles and molecules with chemically functionalized surface and/or nanoparticles conjugated with a wide array of molecules. Chemical functionalization is known in the art of synthesis. See, for example, March, Advanced Organic Chemistry, 6th Ed., Wiley, 2007. Functionalization can be carried out on the surface of the particle, or on both the surface and the interior of the particle, but the structure of the glycogen molecule as a singlebranched homopolymer as described above is maintained.

Such functionalized surface groups include, but are not limited to, nucleophilic and electrophilic groups, acidic and basic groups, including for example carbonyl groups, amine groups, thiol groups, carboxylic or other acidic groups. Amino groups can be primary, secondary, tertiary, or quaternary amino groups. The nanoparticles described herein also can be functionalized with unsaturated groups such as vinyl and allyl groups.

The nanoparticles, as isolated and purified, can be either directly functionalized or indirectly, where one or more intermediate linkers or spacers can be used. The nanoparticles can be subjected to one or more than one functionalization steps including two or more, three or more, or four or more functionalization steps.

Functionalized nanoparticles can be further conjugated with various desired molecules, which are of interest for a variety of applications, such as biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds.

Known methods for polysaccharide functionalization or derivatization can be used. For example, one approach is the introduction of carbonyl groups, by selective oxidation of glucose hydroxyl groups at positions of C-2, C-3, C-4 and/or C-6. There is a wide spectrum of oxidative agents which can be used such as periodate (e.g., potassium periodate), bromine, dimethyl sulfoxide/acetic anhydride (DMSO/Ac₂O) [e.g., U.S. Pat. No. 4,683,298], Dess-Martin periodinane, etc.

The nanoparticles described herein when functionalized with carbonyl groups are readily reactive with compounds bearing primary or secondary amine groups. This results in imine formation which can be further reduced to amine with a reductive agent e.g., sodium borohydrate. Thus, the reduction step provides an amino-product that is more stable than the imine intermediate, and also converts unreacted carbonyls in hydroxyl groups. Elimination of carbonyls significantly reduces the possibility of non-specific interactions of derivatized nanoparticles with non-targeted molecules, e.g. plasma proteins.

The reaction between carbonyl- and amino-compounds and the reduction step can be conducted simultaneously in one vessel (with a suitable reducing agent introduced to the same reaction mixture). This reaction is known as direct reductive amination. Here, any reducing agent, which selectively reduces imines in the presence of carbonyl groups, e.g., sodium cyanoborohydrate, can be used.

For the preparation of amino-functionalized nanoparticles from carbonyl-functionalized nanoparticles, any ammonium salt or primary or secondary amine-containing compound can be used, e.g., ammonium acetate, ammonium chloride, hydrazine, ethylenediamine, or hexanediamine. This reaction can be conducted in water or in an aqueous polar organic solvent e.g., ethyl alcohol, DMSO, or dimethylformamide.

Reductive amination of the nanoparticles described herein can be also achieved by using the following two step process. The first step is allylation, i.e., converting hydroxyls into allyl-groups by reaction with allyl halogen in the presence of a reducing agent, e.g., sodium borohydrate. In the second step, the allyl-groups are reacted with a bifunctional aminothiol compound, e.g., aminoethanethiol.

Amino-functionalized nanoparticles are amenable to further modification. For example, amino groups are reactive to carbonyl compounds (aldehydes and ketones), carboxylic acids and their derivatives, (e.g., acyl chlorides, esters), succinimidyl esters, isothiocyanates, sulfonyl chlorides, etc.

In certain embodiments, the nanoparticles described herein are functionalized using the process of cyanylation. This process results in the formation of cyanate esters and imidocarbonates on polysaccharide hydroxyls. These groups react readily with primary amines under very mild conditions, forming covalent linkages. Cyanylation agents such as cyanogen bromide, and, preferably, 1-cyano-4-diethylamino-pyridinium (CDAP), can be used for functionalization of the nanoparticles.

Functionalized nanoparticles can be directly attached to a chemical compound bearing a functional group that is capable of binding to carbonyl- or amino-groups. However, for some applications it may be important to attach chemical compounds via a spacer or linker including for example a polymer spacer or a linker. These can be homo- or hetero-bifunctional linkers bearing functional groups which include, but are not limited to, amino, carbonyl, sulfhydryl, succimidyl, maleimidyl, and isocyanate e.g., diaminohexane, ethylene glycobis (sulfosuccimidylsuccinate) (sulfo-EGS), disulfosuccimidyl tartarate (sulfo-DST), dithiobis (sulfosuccimidylpropionate) (DTSSP), aminoethanethiol, and the like.

Chemical Compounds and Modifiers for the Nanoparticles/Conjugation

In certain embodiments, chemical compounds which can be used to modify the nanoparticles described herein include, but are not limited to: biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds.

In certain embodiments, biomolecules used as chemical compounds to modify the nanoparticles described herein include, but are not limited to, enzymes, receptors, neurotransmitters, hormones, cytokines, cell response chemical compounds such as growth factors and chemotactic factors, antibodies, vaccines, haptens, toxins, interferons, ribozymes, anti-sense agents, and nucleic acids.

In certain embodiments, small molecule modifiers of the nanoparticles described herein can be those which can be useful as catalysts and include, but are not limited to, metal-organic complexes.

In certain embodiments, pharmaceutically useful moieties used as modifiers for the nanoparticles include, but are not limited to, hydrophobicity modifiers, pharmacokinetic modifiers, biologically active modifiers and detectable modifiers.

In certain embodiments, the nanoparticles can be modified with chemical compounds which have light absorbing, light emitting, fluorescent, luminescent, Raman scattering, fluorescence resonant energy transfer, and electroluminescence properties.

In certain embodiments, diagnostic labels of the nanoparticles include, but are not limited to, diagnostic radiopharmaceutical or radioactive isotopes for gamma scintigraphy and positron emission tomography (PET), contrast agents for Magnetic Resonance Imaging (MRI) (e.g. paramagnetic atoms and superparamagnetic nanocrystals), contrast agents for computed tomography, contrast agents for imaging with X-rays, contrast agents for ultrasound diagnostic methods, agents for neutron activation, and other moieties which can reflect, scatter or affect X-rays, ultrasounds, radiowaves and microwaves, fluorophores in various optical procedures, etc. Diagnostic radiopharmaceuticals include gamma-emitting radionuclides, e.g., indium-111, technetium-99m and iodine-131, etc. Contrast agents for MRI (Magnetic Resonance Imaging) include magnetic compounds, e.g. paramagnetic ions, iron, manganese, gadolinium, lanthanides, organic paramagnetic moieties and superparamagnetic, ferromagnetic and antiferromagnetic compounds, e.g., iron oxide colloids, ferrite colloids, etc. Contrast agents for computed tomography and other X-ray based imaging methods include compounds absorbing X-rays, e.g., iodine, barium, etc. Contrast agents for ultrasound based methods include compounds which can absorb, reflect and scatter ultrasound waves, e.g., emulsions, crystals, gas bubbles, etc. Other examples include substances useful for neutron activation, such as boron and gadolinium. Further, labels can be employed which can reflect, refract, scatter, or otherwise affect X-rays, ultrasound, radiowaves, microwaves and other rays useful in diagnostic procedures. In certain embodiments a modifier comprises a paramagnetic ion or group.

In certain embodiments, two or more different chemical compounds are used to produce multifunctional derivatives. For example, the first chemical compound is selected from a list of potential specific binding biomolecules, such as antibody and aptamers, and then the second chemical compound is selected from a list of potential diagnostic labels.

In certain embodiments, the nanoparticles described herein can be used as templates for the preparation of inorganic nanomaterials using methods that are generally known in the art (see, e.g. Nanobiotechnology II, Eds Mirkin and Niemeyer, Wiley-VCH, 2007.) This can include functionalization of the nanoparticles with charged functional groups, followed by mineralization which may include incubation of functionalized nanoparticles in solutions of various cations, e.g. metals, semiconductors. Mineralized nanoparticles described herein can be then purified and used in various applications, which include but are not limited to medical diagnostics, sensors, optics, electronics, etc.

Compositions

In one embodiment, the nanoparticle composition is in the form of an aqueous extract as obtained after the step of ultrafiltration.

In one embodiment, the nanoparticle composition is dried and the composition is a powder.

Dried nanoparticle compositions of the present invention are easily soluble/dispersible in water, glycerin and in some organic solvents such as dimethyl sulfoxide (DMSO) or dimethylformamide DMF. In one embodiment, the composition comprises the dried nanoparticles dispersed in water or a solvent. The monodisperse nanoparticle compositions have unique rheological properties compared to previous glycogen compositions. Aqueous dispersions of nanoparticle compositions of the present invention show no significant viscosity up to a concentration of 25% by weight. As a comparison, the “pure phytoglycogen” of Yao (WO 2013/019977) shows a viscosity at 15.2 w/w of 3.645 Pas (3645±315 mPas).

In one embodiment, the composition is shelf-stable at room temperature for at least 24 months from the date of manufacture.

### INDUSTRIAL APPLICABILITY

The compositions of monodisperse photoglycogen nanoparticles disclosed herein can be used in a wide range of food, personal care, industrial and medical applications. For example, the compositions can be used as an additive to control rheology, moisture retention and surface properties. Examples of applications include: film forming, low glycemic index source of carbohydrates, texture enhancers, dermal fillers, stabilizer for vitamins and other photosensitive bioactive compounds, pigment extender, medical lubricant and excipient, drug delivery agent. Compositions of the present invention can also be used to improve the UV protection of suncare formulations and to enhance the photostability of bioactives and other photolabile compounds, such as sunscreens, vitamins, and pharmaceuticals.

The monodisperse phytoglycogen nanoparticles disclosed herein are particularly useful as film-forming agents. Because the nanoparticles are monodisperse, uniform close-packed films are possible. The compositions form stable films with low water activity. Water activity characterizes the degree to which a material can bind water and also the degree to which water molecules can migrate within the material. Water activity is important in the food industry, where it is necessary to find a balance between the physical strength of a product, which increases with its dryness, and the taste of a product, which often increases with higher moisture content. Control of water activity is particularly important in food products that contain several structurally different components, e.g. the bulk of a muffin and the icing coating on the top of the muffin. The composition of the present invention can be used as a barrier film between different components of food products. For example, if the food product is relatively dry, a concentrated aqueous solution of the monodisperse phytoglycogen nanoparticles of the present invention can be sprayed onto the surface of a food product component before another component is brought into contact with the first component and allowed to dry. For the case in which the food product already contains a substantial amount of moisture, a fine powder of the phytoglycogen nanoparticles can be sprinkled onto the surface of the first food component until a continuous film is formed, after which the second component is brought into contact. The composition forms a barrier film and substantially reduces diffusion of water molecules from one food component to another. This barrier film forming property can also be used in the manufacturing of drug and vitamin pills, for which diffusion of water between components is not desirable.

In one embodiment, the composition of monodisperse phytoglycogen nanoparticles disclosed herein are used for drug delivery. The monodisperse phytoglycogen nanoparticles are non-toxic, have no known allergenicity, and can be degraded by glycogenolytic enzymes (e.g. amylases and phosphorylases) of the human body. The products of enzymatic degradation are non-toxic, neutral molecules of glucose. The nanoparticles exhibit excellent chemical compound carrying capacity since they can be conjugated with drugs directly or via molecular spacers or tethers. The drug-conjugated nanoparticle can be further modified with specific tissue targeting molecules, such as folic acid, antibodies or aptamers. The low polydispersity allows uniform derivatization and drug distribution, and associated predictable pharmacokinetics. Finally, the compact spherical molecule, neutral charge and highly hydrophilicity are associated with efficient cell uptake.

### Example 1. Extraction of Glycogen (Phytoglycogen) from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to CFF using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

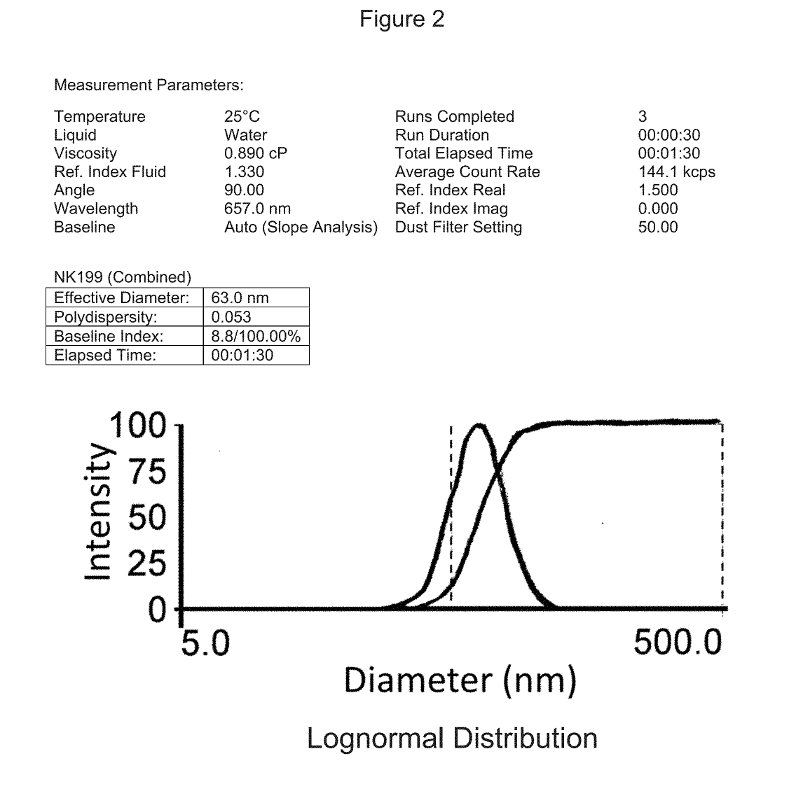

According to DLS measurements, the phytoglycogen nanoparticles produced had particle size diameter of 83.0 nm and the polydispersity index of 0.081 (FIG. 2).

### Example 2

250 g of dry corn kernels of NK199 variety harvested at dent stage were ground to the particle size of less than 0.5 mm. Cold water extraction was performed at 20° C. with moderate agitation for 20 min. Insoluble components were precipitated by centrifugation at 8,000×g. Multistage microfiltration was performed on the supernatant with filtration media pore size of 10.0, 1.0 and 0.1 μm. Cross Flow Filtration (diafiltration) was performed with a MWCO of 300 kDa at RT and diavolume of 6. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried glycogen was 17.5 g.

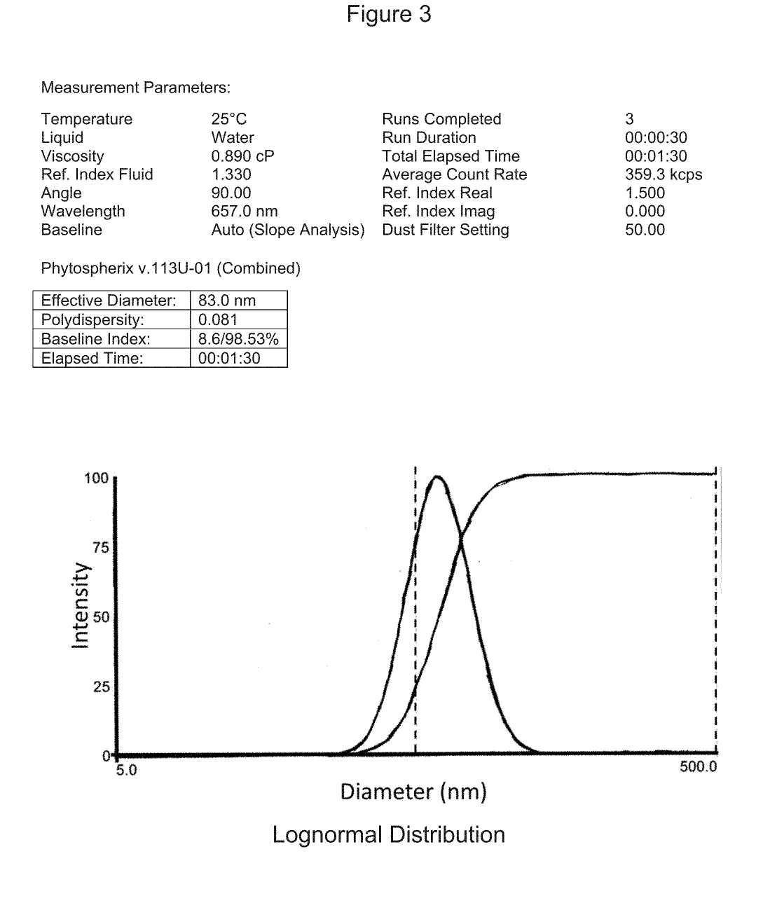

According to DLS measurements, the phytoglycogen nanoparticles produced had particle size diameter of 63.0 nm and a polydispersity index of 0.053 (FIG. 3).

### Example 3. Characterization of Corn Kernel Phytoglycogen Prepared According to Example 2

Phytoglycogen nanoparticles prepared as in Example 2 were characterized by DLS and the results are presented in Table 1. All cultivars are standard (su) type.

| | Yield, % | | |
|---|---|---|---|
| | on kernel | Particle | Poly- |
| | abs dry | size, | dispersity |
| Cultivar* | wt | nm | Index |
| Country Gentlemen | 24.78 | 68.8 | 0.103 |
| Sugar Dots | 28.02 | 69.4 | 0.081 |
| Jubilee | 27.25 | 66.9 | 0.086 |
| Stowell's Evergreen | 27.47 | 66.6 | 0.071 |
| NK199 | 28.46 | 63 | 0.053 |
| Honey and Cream | 32.64 | 68.8 | 0.103 |
| Silver Queen | 27.20 | 68.5 | 0.129 |
| Golden Bantam | 35.71 | 68.1 | 0.098 |
| Quickie | 31.43 | 63.9 | 0.118 |
| Earlivee Yellow | 31.81 | 77.5 | 0.107 |
| Early Sunglow | 23.79 | 69.6 | 0.099 |
| G90 | 29.01 | 67.1 | 0.087 |
| Seneca Horizon | 25.55 | 73.3 | 0.109 |
| Iochieff | 30.11 | 66.5 | 0.107 |
| Butter and Sugar | 30.05 | 75.3 | 0.075 |

The phytoglycogen nanoparticles produced had a polydispersity index between 0.071 and 0.129, with an average polydispersity index of 0.10.

Phytoglycogen nanoparticles prepared as in Example 2 using corn kernels of se and sh type, harvested at the dent stage, were characterized and the results are presented in Table 2.

| | | Yield, | |
|---|---|---|---|
| | | % on | Particle |
| | | kernel | size, |
| Cultivar | Type | dry wt | nm |
| Navajo | se bicolor | 5.4 | 95.2 |
| Welcome | se yellow | 7 | 98.7 |
| Speedy Sweet | se bicolor | 7.2 | 60.3 |
| Fleet Bicolor | se bicolor | 9.5 | 95.1 |
| Head Start | se yellow | 17.3 | 88 |
| Aladdin | se bicolor | 20.4 | 92.1 |
| Sensor | se bicolor | 21.4 | 84.3 |
| Silver King | se white | 25.8 | 88.1 |
| Sensor | se bicolor | 21.1 | 102.8 |
| Delectable | se bicolor | 20.1 | 91.1 |
| Colorow | se yellow | 24 | 100.4 |
| Brocade | se bicolor | 20 | 115 |
| Trinity | se bicolor | 17.6 | 95.8 |
| Temptation | se bicolor | 14.2 | 94.2 |
| Sheba A | sh | 0 | — |
| Gourmet | sh | 0 | — |
| Obsession | | | |
| Gourmet 2281 | sh | 0 | — |
| Devotion | sh | 0 | — |

### Example 4

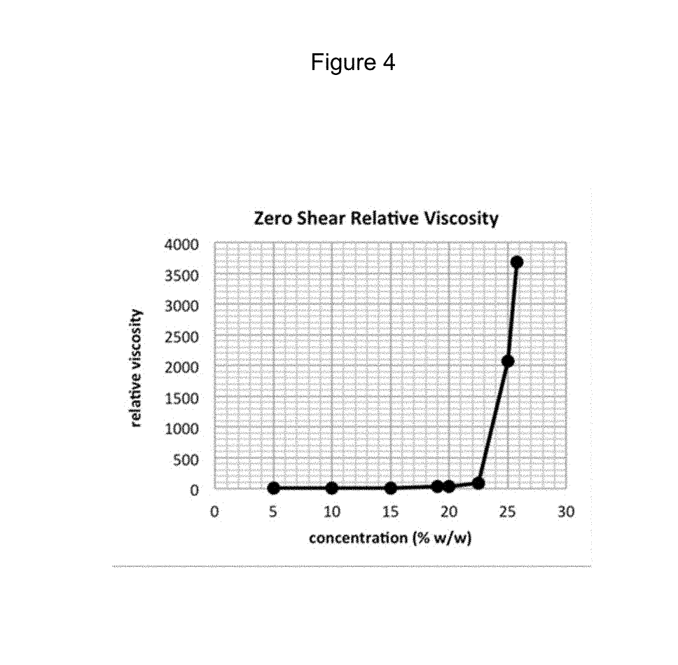

Dried nanoparticle compositions as described herein were dissolved in water at various concentrations from 5 to 30 w/w %. Results are shown in FIG. 4. Solutions provided were clear with no significant viscosity up to concentration of 25% by weight. Viscosity increased significantly for concentration greater than 25% w/w. For concentrations above 20% w/w the solutions showed strong shear thinning properties.

### Example 5

Effects of Milling on Extraction of Phytoglycogen Nanoparticles

In first exemplary method, approximately 150 kg of frozen sweet corn which contained 72.44% moisture content was thawed and milled using a Stephan Microcut set to a 0.5-0.9 mm cutting gap. In a second exemplary method, approximately 100 kg of frozen sweet corn (73.29% moisture content) was gently milled using a Biro Model 6642 at a cutting gap of 5-15 mm.

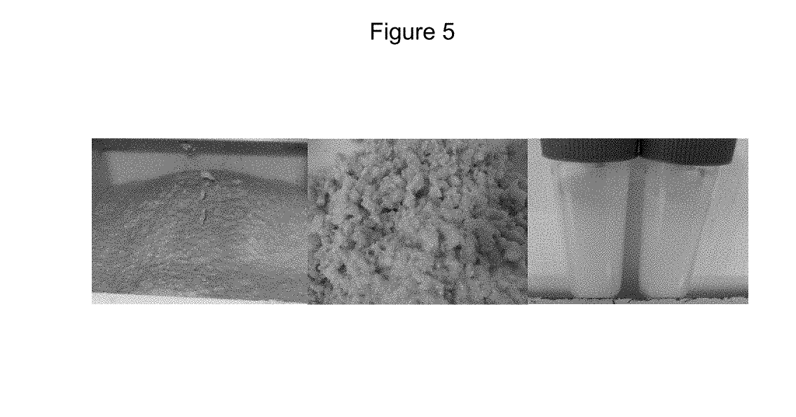

Based on the extent of milling and the type of equipment used, the pulverized kernel size ranged between 1-15 mm (FIG. 5). In instances where the kernel is finely milled, an emulsification of the kernel's protein and lipid fractions can occur with the glycogen fraction which negatively impacts the purity of the final product.

### Example 6

Aqueous Extraction of Phytoglycogen Nanoparticles from Sweet Corn Kernels

Pulverized corn kernels prepared as in Example 5 were mixed in a 1:1-4 ratio of deionized water for a period of 10-60 min to extract the majority of the phytoglycogen fractions from the endosperm of the kernel. Surprisingly, the amount of phytoglycogen extracted past a ratio of 1:2 (solid to liquid) yields the same amount of phytoglycogen (aprox. 40% per dry weight basis). Furthermore, an incubation exceeding 30 min results into maximum yield of phytoglycogen nanoparticles extracted out of the sweet corn kernels.

### Example 7

Effects of Temperature Control During Extraction of Phytoglycogen Nanoparticles

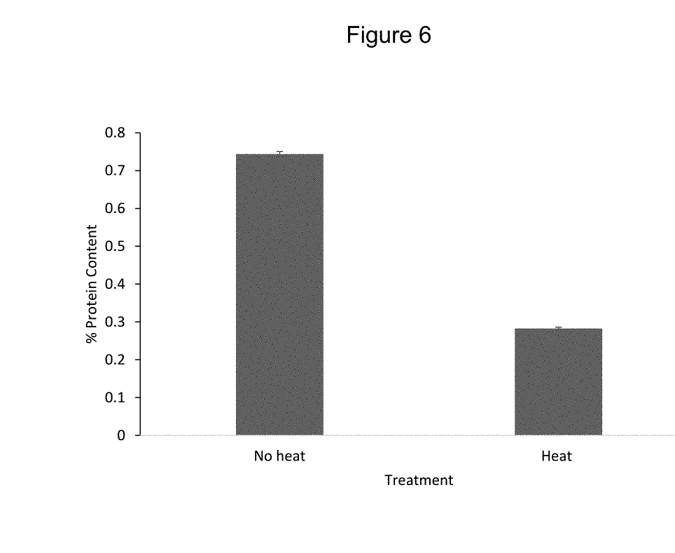

The extraction of phytoglycogen as described in Example 6 was performed at room temperature (10° C.) and at temperatures between 20-80° C. It was observed that the maximum amount of extraction resulted in temperatures ranging between 50 and 70° C. and surprisingly the level of purity of pytoglycogen improved accordingly as shown in FIG. 6.

### Example 8

Effects of pH on Extraction of Phytoglycogen Nanoparticles

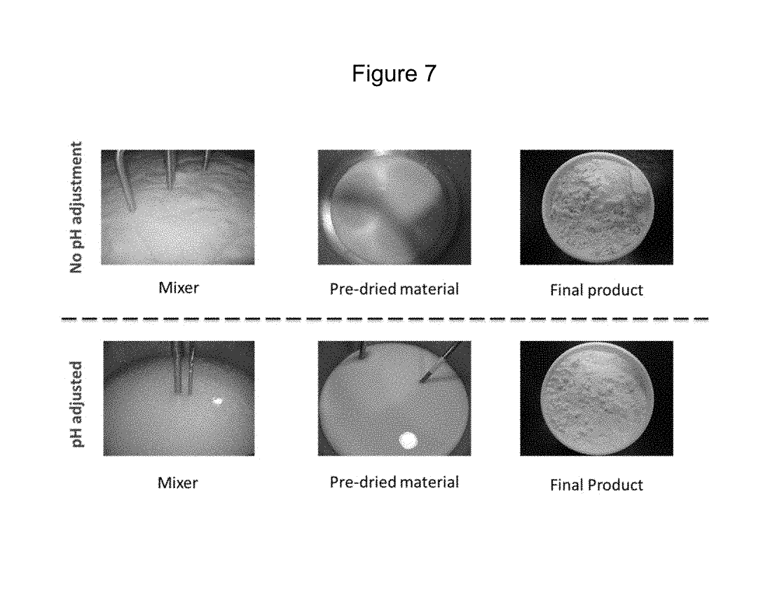

A corn slurry as described in Example 7 was subjected to a pH range of 3-11 and the effects of this treatment on the overall coloration of the extracted nanoparticles was evaluated (FIG. 7). Phytoglycogen nanoparticles produced from non-acidified corn juice were yellower in colour compared to those produced with a mild acidic treatment as evaluated in Example 9.

### Example 9

Color Characterization of pH Controlled Extraction of Phytoglycogen Nanoparticles

Phytoglycogen nanoparticles prepared as in Example 8 were characterized by colorimetric measurements and the results appear in Table 3. Colorimetric measurements are based on the L*, a* and b* parameters, where the L* value measures the degree of whiteness/darkness and the higher the L* value, the lighter the colour. The a* value indicates the balance between redness and greenness of the phytoglycogen nanoparticles with positive (+) value (a>0) corresponding to red colour and negative (−) value (a<0) to green. The b* value indicates the balance between yellowness (+) (b>0) and blueness (−) (b<0). Generally, a sample containing high residual protein content tends to be yellower in colour (higher b value) as seen in non-pH adjusted samples. This is due to the presence of the residual zein, which has a beta-carotene molecule imbedded in its alpha helical structure.

TABLE 3
Colorimetric measurements of powdered phytoglycogen nanoparticles

| Sample | L* | a* | b* |
|---|---|---|---|
| No pH adjustment | 91.73 ± 0.02 | 1.02 ± 0.01 | 18.04 ± 0.02 |
| pH adjusted | 95.42 ± 0.01 | −0.50 ± 0.02 | 2.72 ± 0.02 |

### Example 10

Particle Size Characterization of pH Controlled Extraction of Phytoglycogen Nanoparticles

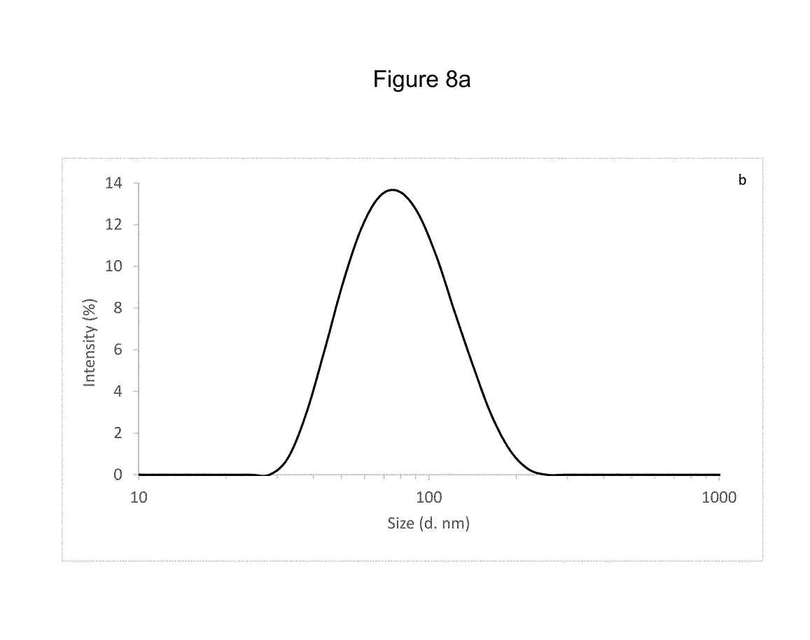

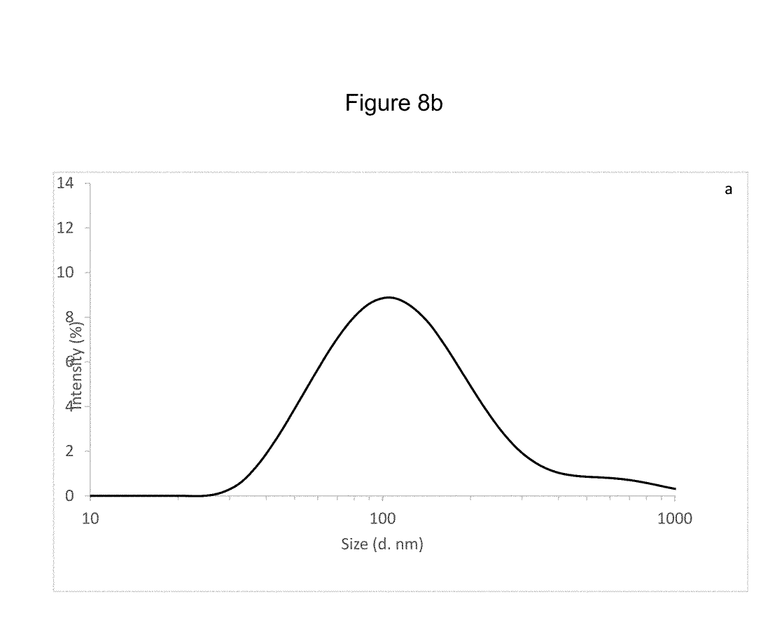

Phytoglycogen nanoparticles prepared as in Example 8 were characterized by DLS measurements and the results as shown in FIG. 8a, where pH is unadjusted. Where pH is unadjusted the particle size distribution is wider, bimodal and the average diameter of the particle is greater than when mildly acid treated as show in FIG. 7b. This is attributed to the presence of hydrophobic proteinaceous moieties that tend to agglomerate together in aqueous environment. Mildly acidifying the corn slurry promotes better separation of the proteinaceous fraction resulting in cleaner phytoglycogen nanoparticles with particle size ranging between 70-80 nm according to DLS measurements (FIG. 8b).

### Example 11

Protein Content Determination of pH Controlled Extraction of Phytoglycogen Nanoparticles

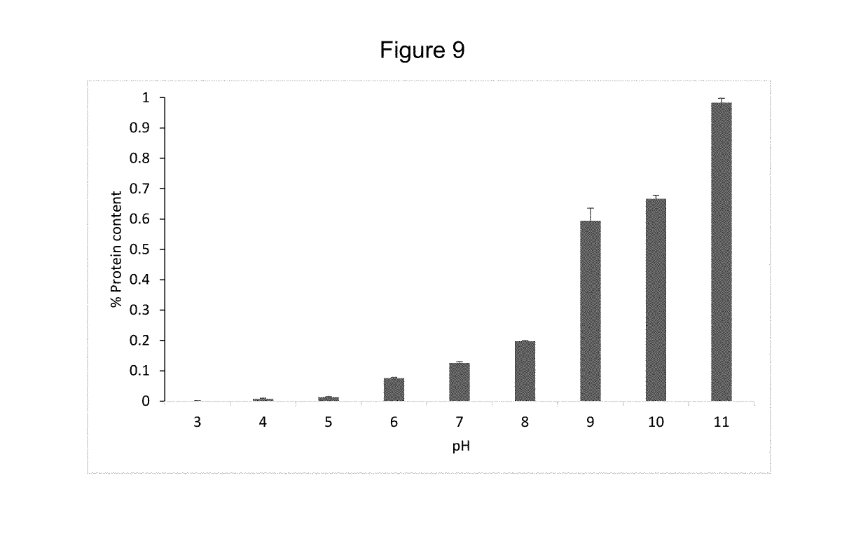

Phytoglycogen nanoparticles prepared as in Example 8 were characterized by Bradford assay method for quantification of residual protein content (FIG. 9). It is evident that mildly acidifying the corn juice diminishes residual proteins present in phytoglycogen nanoparticles. As reported by those familiar in the art of protein chemistry, zein, the principal protein found in sweet corn, undergoes deamidation of its most abundant amino acid, glutamine to form glutamic acid. Protonation of acidic amino acid in low pH condition increases hydrophobicity of zein. Deamidated zein with higher hydrophobicity induces higher protein-protein interactions, resulting in lower affinity with its aqueous environment and promotes zein moieties to coagulate in the acidic solution [18-19]. As the protein fraction becomes coagulated, it eases its separation from the aqueous environment and is readily discarded in Step c.

### Example 12

Lipid Content Determination of pH Controlled Extraction of Phytoglycogen Nanoparticles

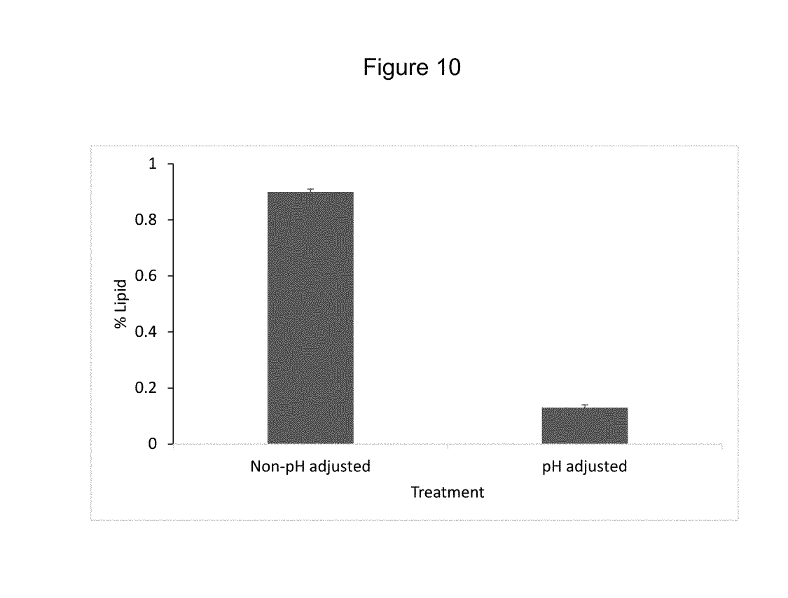

Phytoglycogen nanoparticles prepared as in Example 8 of the present invention were characterized by Swedish tube method as described by [20] for quantification of residual lipid content and the results appear in FIG. 10. It is evident that mildly acidifying the corn slurry reduces the lipid content of the phytoglycogen nanoparticles. Without wishing to be bound by a theory, it is hypothesized that the lipid moieties form hydrophobic interactions with the native zein (major hydrophobic protein fraction of corn) and separate out at Step c.

### Example 13

Reducing Sugar Content Determination of pH Controlled Extraction of Phytoglycogen Nanoparticles

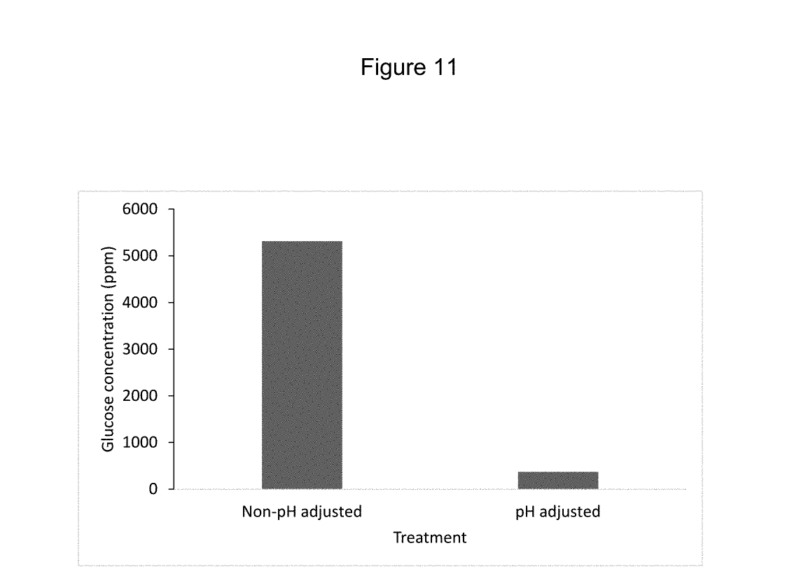

Phytoglycogen nanoparticles prepared as in Example 8 were characterized by potassium ferricyanide colorimetric assay for quantification of reducing sugar content and the results appear in FIG. 11. It is apparent that by acidifying the corn juice, a decrease in the reducing sugar content of the phytoglycogen nanoparticles occurs. It is surmised that mild acidic pH environment promotes dissociation of the reducing sugars from the glycogen fraction, which becomes easily removed during solid-liquid separation step as well as the filtration step.

### Example 14

Size Exclusion Characterization of pH Controlled Extraction of Phytoglycogen Nanoparticles

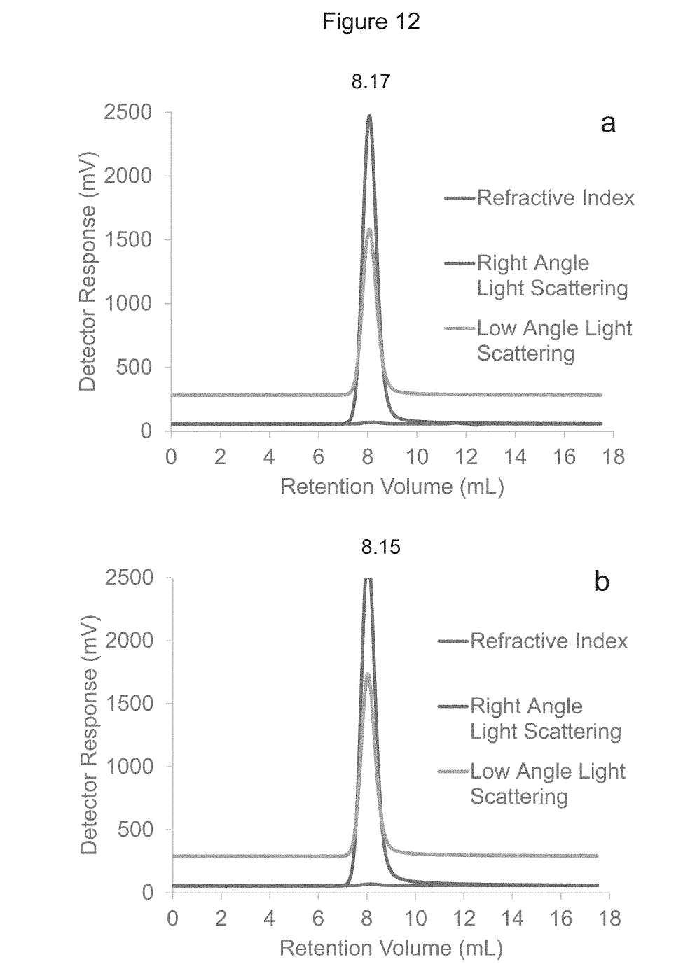

Phytoglycogen nanoparticles prepared as in Example 8 were characterized by SEC-HPLC to determine if incorporation of a mild acidic treatment in Step (b) induces hydrolysis of phytoglycogen nanoparticles and the results appear in FIG. 12. It is evident by the SEC chromatogram that mildly acidifying the corn juice (FIG. 12b) does not induce a breakdown of the native glycogen structure as noted in FIG. 12a. A breakdown of the native glycogen fraction would have been apparent in the chromatogram at a higher elusion volume.

### REFERENCES

1. Manners, Carbohydrate Polymers, 16 (1991) pp. 37-82.
2. Pflüger, 1894, Archly. für Physiologie, pp 394-396.
3. Somogyi, 1934, J. Biol. Chem., 104:245-253.
4. Stetten et al., 1956. J. Biol. Chem. 222, 587-599.
5. Bell and Young, 1934, Biochem. J. 28:882-890.
6. Orell et al., 1964, J. Biol Chem., 239: 4021-4026.
7. Bueding and Orrell. J. Biol Chem. 1961, 236: 2854-7.
8. Huang and Yao, Carbohydrate Polymers, 2011, 83: 1165-1171.
9. Meléndez-Hevia et al., (1993) Optimization of molecular design in the evolution of metabolism: the glycogen molecule, Biochem. J. 295: 477-483.
10. Meléndez et al., (1997) How did glycogen structure evolve to satisfy the requirement for rapid mobilization of glucose? A problem of physical constraints in structure building. J. Mol. Evol. 45:446-455.
11. Meléndez et al., (1998) Physical constraints in the synthesis of glycogen that influence its structural homogeneity: a two-dimensional approach. Biophys. J. 75: 106-114.
12. Meléndez et al., (1999) The fractal structure of glycogen: a clever solution to optimize the cell metabolism. Biophys. J. 77:1327-1332.
13. DiNuzzo M. (2013) Kinetic analysis of glycogen turnover: relevance to human brain (13) C-NMR spectroscopy. Journal of cerebral blood flow and metabolism 33:1540-1548.
14. Scheffler, S. L., Wang, X., Huang, L., San-Martin Gonzalez, F., & Yao, Y. (2010). Phytoglycogen octenyl succinate, an amphiphilic carbohydrate nanoparticle, and ε-polylysine to improve lipid oxidative stability of emulsions. Journal of Agricultural and Food Chemistry, 58, 660-667.
15. Shin, J., Simsek, S., Reuhs, B., & Yao, Y. (2008). Glucose release of water-soluble starch-related α-glucans by pancreatin and amyloglucosidase is affected by the abundance of α-1,6 glucosidic linkages. Journal of Agricultural and Food Chemistry, 56, 10879-10886.
16. Thompson, D. B. (2000). On the non-random nature of amylopectin branching. Carbohydrate Polymers, 43, 223-239.
17. Yun, S. H., &Matheson, N. K. (1993). Structures of the amylopectins of waxy, normal, Amylase-extender, and wx: Ae genotypes and of the phytoglycogen of maize. Carbohydrate Research, 243, 307-321.
18. Zhang, B., Luo, Y., Wang, Q. (2011). Effect of acid and base treatments on structural, rheological and antioxidant properties of α-zein. Food Chemistry, 124, 210-220.
19. Cabra, V., Arreguin, R., Vazquez-Duhalt, R., Farres, A. (2007). Effect of alkaline deamidation on the structure, surface ydrophobilicy, and emulsifying properties of the Z19 alpha-zein. Journal of Agricultrural and Food Chemistry, 55, 439-445.
20. Troeng, S. (1955). Oil determination of oilseed. Gravimetric routine method. Journal of the American Oil Chemists' Society, 32, 124-126.

1. A process for producing monodisperse phytoglycogen nanoparticles comprising:
a. subjecting disintegrated phytoglycogen-containing plant material to a water treatment at a temperature equal to or less than about 70° C.;
b. adjusting the pH of the product of step (a.) to between about 3.5 and about 7;
c. subjecting the product of step (b.) to a solid-liquid separation to obtain an aqueous extract; and
d. subjecting the aqueous extract from step (c.) to ultrafiltration to remove impurities having a molecular weight of less than about 300 kDa to obtain an aqueous composition comprising monodisperse phytoglycogen nanoparticles.

2. The process of claim 1, wherein the phytoglycogen-containing plant material is corn kernels.

3. The process of claim 2, wherein the corn kernels are frozen.

4. The process of claim 2, wherein the phytoglycogen-containing plant material is standard type (su) or surgary extender (se) type sweet corn.

5. The process of claim 4, wherein the phytoglycogen-containing plant material is milk stage or dent stage kernel of standard type (su) or surgary extender (se) type sweet corn.

6. The process of claim 1 comprising subjecting the aqueous composition comprising monodisperse phytoglycogen nanoparticles of step (d.) to enzymatic treatment using amylosucrose, glycosyltransferase, branching enzymes or any combination thereof.

7. The process of claim 1 wherein the ultrafiltration of step (d.) removes impurities having a molecular weight less than about 500 kDa.

8. The process of claim 1, further comprising (d1) passing the aqueous extract of step (c.) through microfiltration material having a maximum average pore size between about 10 μm and about 40 μm

9. The process of claim 8, further comprising adding an adsorptive filtration aid prior to step (d.) or step (d1.).

10. The process of claim 9 wherein the adsorptive filtration aid is a diatomaceous earth.

11. The process of claim 1, wherein the solid-liquid separation comprises agitating the product of step (b.) for a period of 30 to 60 minutes.

12. The process of claim 1 wherein the pH is adjusted using NaOH.

13. The process of claim 1 wherein the pH is adjusted using one or more of phosphoric acid, acetic acid, citric acid, sulfuric acid and hydrogen chloride.

14. The process of claim 1, further comprising (e.) drying the aqueous composition comprising monodisperse phytoglycogen nanoparticles to yield a dried composition of substantially monodisperse phytoglycogen nanoparticles.

15. A composition produced according to the process of claim 1 comprising phytoglycogen nanoparticles obtained from a phytoglycogen-containing plant material, the phytoglycogen nanoparticles having a polydispersity index of less than 0.35 as measured by dynamic light scattering (DLS).

16. The composition of claim 15, wherein the phytoglycogen nanoparticles have an average particle diameter of between about 30 nm and about 150 nm as measured by DLS.

17. The composition of claim 16, wherein the phytoglycogen nanoparticles have an average particle diameter between about 50 nm and 120 nm as measured by DLS.

18. The composition of claim 17, wherein the physoglycogen nanoparticles have an average particle diameter between about 60 nm and 80 nm as measured by DLS.

19. The composition of claim 15 wherein the phytoglycogen nanoparticles have a total protein content of 2-3% (w/w) as measured by the Bradford assay.

20. The composition of claim 15 wherein the phytoglycogen nanoparticles have a total protein content of less than 0.1% (w/w) as measured by the Bradford assay.

21. The composition of claim 15 wherein the phytoglycogen nanoparticles have a reducing sugar content of less than 0.15% as measured by the potassium ferricyanide calorimetric assay.

22. The composition of claim 15 wherein the composition is a powder.

23. The composition of claim 15 wherein the composition is an aqueous dispersion of the phytoglycogen nanoparticles.

24. A composition produced according to the process of claim 2, comprising phytoglycogen nanoparticles obtained from a phytoglycogen-containing plant material, the phytoglycogen nanoparticles having a polydispersity index of less than 0.35 as measured by dynamic light scattering (DLS).

25. A composition produced according to the process of claim 3, comprising phytoglycogen nanoparticles obtained from a phytoglycogen-containing plant material, the phytoglycogen nanoparticles having a polydispersity index of less than 0.35 as measured by dynamic light scattering (DLS).

26. A composition produced according to the process of claim 4, comprising phytoglycogen nanoparticles obtained from a phytoglycogen-containing plant material, the phytoglycogen nanoparticles having a polydispersity index of less than 0.35 as measured by dynamic light scattering (DLS).

27. A composition produced according to the process of claim 5, comprising phytoglycogen nanoparticles obtained from a phytoglycogen-containing plant material, the phytoglycogen nanoparticles having a polydispersity index of less than 0.35 as measured by dynamic light scattering (DLS).

28. A composition produced according to the process of claim 6, comprising phytoglycogen nanoparticles obtained from a phytoglycogen-containing plant material, the phytoglycogen nanoparticles having a polydispersity index of less than 0.35 as measured by dynamic light scattering (DLS).

29. A composition produced according to the process of claim 7, comprising phytoglycogen nanoparticles obtained from a phytoglycogen-containing plant material, the phytoglycogen nanoparticles having a polydispersity index of less than 0.35 as measured by dynamic light scattering (DLS).

30. A composition produced according to the process of claim 8, comprising phytoglycogen nanoparticles obtained from a phytoglycogen-containing plant material, the phytoglycogen nanoparticles having a polydispersity index of less than 0.35 as measured by dynamic light scattering (DLS).

31. A composition produced according to the process of claim 9, comprising phytoglycogen nanoparticles obtained from a phytoglycogen-containing plant material, the phytoglycogen nanoparticles having a polydispersity index of less than 0.35 as measured by dynamic light scattering (DLS).

32. A composition produced according to the process of claim 10, comprising phytoglycogen nanoparticles obtained from a phytoglycogen-containing plant material, the phytoglycogen nanoparticles having a polydispersity index of less than 0.35 as measured by dynamic light scattering (DLS).

33. A composition produced according to the process of claim 11, comprising phytoglycogen nanoparticles obtained from a phytoglycogen-containing plant material, the phytoglycogen nanoparticles having a polydispersity index of less than 0.35 as measured by dynamic light scattering (DLS).

34. A composition produced according to the process of claim 12, comprising phytoglycogen nanoparticles obtained from a phytoglycogen-containing plant material, the phytoglycogen nanoparticles having a polydispersity index of less than 0.35 as measured by dynamic light scattering (DLS).

35. A composition produced according to the process of claim 13, comprising phytoglycogen nanoparticles obtained from a phytoglycogen-containing plant material, the phytoglycogen nanoparticles having a polydispersity index of less than 0.35 as measured by dynamic light scattering (DLS).

36. A composition produced according to the process of claim 14, comprising phytoglycogen nanoparticles obtained from a phytoglycogen-containing plant material, the phytoglycogen nanoparticles having a polydispersity index of less than 0.35 as measured by dynamic light scattering (DLS).
